(19) United States
(12) Patent Application Publication
(10) Pub. No.: US 2009/0062677 A1
(43) Pub. Date: Mar. 5, 2009

(54) Method of Recording and Saving of Human Soul for Human Immortality and Installation for it

(76) Inventor: Alexander Alexandrovich Bolonkin, Brooklyn, NY (US)

Correspondence Address: Alexander Bolonkin, 1310 Avenue R, Apt.6-F,, Brooklyn, NY, 11229, US

(21) Appl. No.: 11/613,380

(22) Filed: Dec. 20, 2006

(51) Int. Cl. A61B 5/04; A61B 5/00

(52) U.S. Cl. 600/544; 600/300

(57) ABSTRACT

The inventor shows that a human soul is only the information in human brain. He offers a method for re-writing the human brain on electronic chips. This method allows for the modeling of a human soul in order to achieve man immortality. This method does not damage the brain but works to extend and enhance it.

This method of writing and saving of Human Soul includes: writing of views which person see in during his life by a micro video recorder to a portable memory; writing of sounds which person speaks and hears by microphone; writing of person physical conditions by micro sensors; writing of time and dates; writing of person body position and so on.

An Installation utilized the Method is comprising devices: micro video camera located at man head, microphone, computer (chip) for data processing, portable memory located at an person body and connected to the portable memory; portable recorder for writing of life film located at the man body and connected to the computer and to the portable memory; micro sensors for writing of person physical and environment conditions; a clock for writing of time, the micro sensors for writing of men body positions; navigation system, long time memory high capability storage and so on.

### CROSS-REFERENCE TO RELATED APPLICATIONS

USA PTO disclosure document No. 567484 of Dec. 29, 2004

### STATEMENT REGARDING FEDERALLY SPONSORED RESEARCH OR DEVELOPMENT

Not Applicable

### REFERANCE TO A “SEQUENCE LISTING”

Not Applicable

### REFERENCE TO A MICROFICHE APPENDIX

Not Applicable

### BACKGROUND OF THE INVENTION 1. Field of the Invention

The inventor shows that human soul is only the information in human brain. He offers a new method for re-writing the human brain on electronic chips. This method allows for the modeling of a human soul in order to achieve immortality. This method does not damage the brain but works to extend and enhance it.

2. Description of the Related Art

Immortality is the most cherished dream and the biggest wish of any person. People seldom think about it while they are still young, healthy, and full of energy. But when they get some incurable disease or become old, then there is no bigger wish for them than to live longer, put off the inevitable end. And no matter what heavenly existence in the after-life is promised to them by religion, the vast majority of people want to stay and enjoy life here, on Earth, as long as possible. The inventor shows a real immortality can be only electronically.

### Medical Science and the Issue of Immortality

A great many of doctors and scientists are currently working on the problems of health and longevity. Substantial means are spent on it, about 15-25% of all human labor and resources. There are certain achievements in this direction: we have created wonderful medications (e.g. antibiotics); conquered many diseases; learnt to transplant human organs; created an artificial heart, kidneys, lungs, limbs; learnt to apply physiological solutions directly into the blood stream, and to saturate blood with oxygen. We have gotten inside the most sacred organ—the human brain, even inside its cells. We can record their signals, we can agitate some parts of the brain by electric stimuli inducing a patient to experience certain sensations, images, and hallucinations.

We can attribute the fact that the average life span has increased two times in the last two hundred years to the achievements of modern medicine.

But can medical science solve the problem of immortality? Evidently, it cannot. It cannot do that in principle. This is a dead-end direction in science. Maximum it can achieve is increasing the average life expectancy another 5-10 years. An average person will be expected to live 80 years instead of 70. But what kind of person will it be? A very old one, capable of only existing and consuming, whose medical and personal care will demand huge funds.

The proportion of the elderly and retirees has increased steeply in the last 20-30 years and continues to grow depleting the pension funds and pressuring the younger generation to support them. So it is hard to say whether the modern success of medicine is a blessing or a curse from the point of view of the entire humankind, even though it is definitely a blessing from the point of view of a separate individual.

Humanity as a whole, as a civilization, needs active, able to work and creative members, generating material wealth and moving forward technology and science, not the elderly retirees with their numerous ailments and a huge army of those tending to them. It dreams not of the immortality of an old person, but of the immortality of youthfulness, activity, creativity, enjoying life.

Now there are signs of a breakthrough, but not in the direction the humankind has been working on all along, since the times of the first sorcerers to modern-day highly-educated doctors. Striving to prolong his biological existence, man has been chiseling, so to speak, at the endless stone wall. All he has been able to accomplish is only a dent in that wall—increased life expectancy, conquering some diseases, relieving suffering. As a payoff, the humanity has received a huge army of pensioners and retirees and gigantic expenditure on their upkeep.

Of course, one can continue chiseling at the dent in the wall further on, make it somewhat bigger, aggravating side effects. But we are already approaching the biological limit, when the cause of death and feeblemindedness is not a certain disease which can be conquered, but general deterioration of the entire organism, its decay on the cellular level, when the cells stop to divide. A live cell is a very complex biological formation. In its nucleus it has DNA—biological molecules consisting of tens of thousands of atoms connected between themselves with very fragile molecular links. Suffice it to say, that temperature fluctuation of only a few degrees can ruin these links. That is why a human organism maintains a certain temperature—36.7 C. Raising this temperature only 2-3 degrees causes pain, and 5-7 degrees leads to death. Maintaining the existence of human cells also presents a big problem for humanity involving food, shelter, clothes and ecologically clean environment.

Nevertheless, human cells cannot exist eternally even under ideal conditions. This follows from the atomic-molecular theory. Atoms of biological molecules permanently oscillate and interact with each other. According to the theory of probability, sooner or later the impulses of adjacent atoms influencing the given atom, add up, and the atom acquires enough speed to break loose from its atomic chain, or at least to transfer into the adjacent position (physicists say that the impulse received by the atom has surpassed the energy threshold which retains the atom in its particular place in the molecular chain). It also means that the cell containing this atom has been damaged and cannot any longer function normally. Thus, for example, we get cancer cells which cannot fulfill their designated functions any more and begin to proliferate abnormally fast and ruin human organs.

This process accelerates manifold when a person has been exposed to a strong electromagnetic radiation, for instance, Roentgen or X-rays, a high-frequency electric current or radioactive materials.

Actually, the process of deforming of the hereditary DNA molecule under the influence of weak cosmic rays can take place from time to time, leading sometimes to birth defects, or it may turn out to be useful for the survival properties. And this plays a positive role for a particular species of plants or animals contributing to their adaptability to the changed environment and their survival as a species. But for a particular individual such aberration is a tragedy as a rule, since the overwhelming majority of such cases are birth defects, with only few cases of useful mutations. And human society in general is suspicious of people who are radically different in their looks or abilities.

### BRIEF SUMMARY OF THE INVENTION

The inventor shows that a human soul is only the information in human brain. He offers a method for re-writing the human brain on electronic chips. This method allows for the modeling of a human soul in order to achieve immortality. This method does not damage the brain but works to extend and enhance it.

This method of writing and saving of Human Soul includes: writing of views which person see in during his life by micro video recorder to portable memory; writing of sounds which person speaks and hears by microphone; writing of person physical conditions by micro sensors; writing of time and dates; writing of person body position, etc.

An Installation used the Method comprising devices: portable memory located at an person body; computer (chip) for data processing located at the person body and connected to the portable memory; micro camcorder (micro video camera) for writing of life film located at the person body and connected to the computer and to the portable memory; microphone for writing all sounds, which person speaks and hears, located at the person body and connected to said computer and to said portable memory; micro sensors for writing of person physical conditions located at the person body and connected to the computer and to the portable memory; micro clock for writing of time located at the person body and connected to the computer and to the portable memory; micro sensors for writing of person body positions located at the person body and connected to the computer and to the portable memory; long time memory high capability storage.

Writing of human soul is absolutely new idea of modeling, re-writing, and saving of human soul and using it for person immortality. No any materials about this method and installation.

### BRIEF DESCRIPTION OF THE SEVERAL VIEWS OF THE DRAWINGS

FIG. 1. Principal scheme of the installation (equipment) of writing human soul (full information about human life) for man immortality. The cells contain:
1) micro devices (micro video camera, microphone, computer (chip), portable recorder, portable power source, portable memory, etc.);
2) micro sensors and devices for writing of brain impulses, oscillation, fluctuation and a state of man health and person physical conditions (for example: sensors of blood press, heart palpitation, skin resistance, perspiration, skin temperature, respiration activity, mouth water, chill, rigor-like tremor, catarrh, muscle stress, blood mixture, pain, brain oscillation and so on);
3) micro sensors for writing of body positions (for example: sensors of motion, acceleration, distance, angle, mutual position, and so on);
4) devices for measure of outer environment conditions about man such as air temperature, pressure, humidity, gas composition, smells, wind, illumination, radiation, and so on;
5) devices of a portable navigation system for measure of a person position at Earth surface (for example, GPS navigation system);
6) emergency 911 communication;
7) portable power source (for example: chemical, solar, nuclear batteries, and so on);

FIG. 2. Re-writing the information from the portable memory to the high capability long-term storage,

FIG. 3. Location of video camera at human head (forehead).

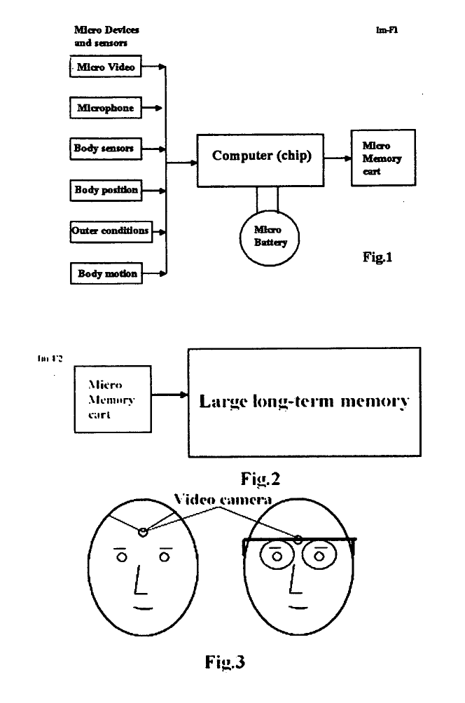

### DETAILED DESCRIPTION OF THE INNOVATION

### 1. Brief Description of Previous Works by the Inventor

In a series of articles (see referenced list at the end) the inventor shows that the purpose of Nature is to create Super Intelligence (SI). With its ability to understand the Universe, advanced entities with SI Power will be able to survive major cataclysms. There is the Law of Increasing Complexity (in opposition to the Entropy Law—increasing chaos). This Law created biological intelligence (people). Human have since became a sovereign entity on the Earth and in Nature above all other creatures.

### 2. Electronic Immortality. Advantages of Electronic Existence

In the noted works the author has shown that the problem of immortality can be solved only by changing the biological human into an artificial form. Such an immortal person made of chips and super-solid material (the E-man, as was called in his articles) will have incredible advantages in comparison to conventional people. An E-man will need no food, no dwelling, no air, no sleep, no rest, and no ecologically pure environment. His brain will work from radio-isotopic batteries (which will work for decades) and muscles that will work on small nuclear engines. Such a being will be able to travel into space and walk on the sea floor with no aqualungs. He will change his face and figure. He will have super-human strength and communicate easily over long distances to gain vast amounts of knowledge in seconds (by re-writing his brain). His mental abilities and capacities will increase millions of times. It will be possible for such a person to travel huge distances at the speed of light. The information of one person like this could be transported to other planets with a laser beam and then placed in a new body.

Such people will not be awkward robots as in the movies. An artificial person will have the opportunity to choose his or her face, body and skin. It will also be possible for them to reproduce and then avoid any period of adolescence including the need for education. It will be impossible to destroy this entity with any kind of weapons, since it will be possible to copy the information of their minds and than keep such information backed up in separate distant locations. As was written in the science fiction book, “The Price of Immortality”, by Igor Getmansky (Moscow, Publish House ECSMO, 2003, Russian) an artificial person will have all of these super-human abilities.

### 3. What are Men and Intelligent Beings

All intelligent creatures have two main components: 1. Information about their environment, about their experience of interacting with nature, people, society (soul) and 2. Capsule (shell), where this information is located (biological brain, body). The capsule supports existence and stores information and programs for all of its operations. The capsule also allows the creature to acquire different sensory information (eyes, ear, nose, tongue and touch) and it moves to different locations in order to interact with the environment.

The main component of an intelligent being is information (soul). The experiences and knowledge accumulated in the soul allows the entity to interact more efficiently in nature in order to survive. If the being has more information and better operational programs (ability to find good solutions), then it is more likely thrive.

For an intelligent being to save its soul it must solve the problem of individual immortality. Currently man creates a soul for himself by acquiring knowledge from parents, educational systems, employment and life experiences. When he dies, most knowledge is lost except for a very small part which is left through works, children and apprentices. Billions of people have lived on Earth, however, we know comparatively little about ancient history. Only after the invention of written language did people have the capacity to easily save knowledge and pass it on to the next generation.

As discussed earlier, the biological storage (human brain) of our soul (information) is unreliable. The brain is difficult to maintain and requires food, lodging, clothes, a good environment and education, etc. To support the brain and body, humans spend about 99% of their time and energy, and eventually what knowledge is gained is taken to the grave in death.

There is only one solution to this problem—re-write all of the brain information (our soul) in more strongly based storage. We must also give the soul the possibility to acquire and manipulate information from the world. This means we must give sensors to the soul so it may have communication and contact with people and other intelligent beings. We must give the soul a mobile system (for example, legs), systems for working (hands), etc. thus giving the soul a new body in which to LIVE.

The reader may ask—these ideas seem interesting, but how does one we re-write a human soul to live within a new carrier, for example, in electronic chips?

### 4. The Main Problem With Electronic Immortality—Re-Writing Brain Information (Soul) to Electronic Chips is That it's Impossible to do This With Current Technology

At present scientists are working to solve this problem. They know that the brain has about 15 billion neurons, and every neuron has about ten connections to neighboring neurons. Neurons gain signals from neighboring neurons, produce signals and then send these signals to others neurons. As a result, humans are able to think and find solutions. On the bases of this way of thinking, humans can come to solutions without exact data. (Concepts of brain were described in my previous articles. For example, see “Locate God in Computer-Internet Networks” or “Science, Soul, Heaven and Supreme Mind”. See also my articles on the Internet and references at end of this article.).

Scientists are learning how to take individual neurons on micro-electrodes and record their impulses. The ideas of scientists are very simple—study how single neurons and small neuronal network work and then model them by computer. They hypothesize that if we can model 15 billion neurons in a computer they will learn how the brain works, and then they will have Artificial Intelligence equaling the human brain.

In my previous work I show this as a dead-end direction for Human Immortality. It's true that we'll create an Artificial Intelligence (AI) that will be more powerful than the human mind. However, it will be HIS AI, and a NEW entity altogether. Our purpose is focused on preserving the CONCRETE PERSON now (more exactly—his SOUL) in a new body in order to achieve immortality.

Why is it impossible to directly write the information of the human brain onto a chip? Because the human brain is constantly changing and neurons permanently change their states. Imagine you want to record the state of a working computer chip. The chip has millions of logical elements which change their state millions of times per second. It is obvious that if you write in series (one after other) the current state of the chip (it is impossible to instantly write ALL states of the chip's elements). To instantly write all neurons one would need to insert a microelectrode into EVERY neuron, this would destroy the human brain before the writing was complete.

I offered another method for the solution of the Main Problem of Immortality.

### 5. Modeling of Soul for a Concrete Person

As said, straight re-writing of a human mind (human soul) to chips is very complex. Straight re-writing is not possible in the near future. All scientific works studying the work of human brains at the present time are useless for the main problem of immortality. They are also unworkable for the problem of artificial intelligence (AI) in the near term, because the brain solves problems by way of general estimations. AI solves problems based on more exact computation and logical data.

To solve the Main Problem of Immortality (MPI) the author offers a method of “MODELLING SOUL” of a concrete person. This method does not require interventions into the brain of a given person. This method may be applied IMMEDIATELY at the present time. But an accurate modeling is needed depending on the modeling period.

Before describing this method, let us analyze the human soul and what components are important for each person and his environment. All information in the human brain (soul) may be separated in two unequal groups: 1. the Memory (permanent knowledge) about the person's life (all that has been seen, heard, made, felt, people which he has met, his (her) behaviors, opinions, wishes, dreams, programs of activity, etc.), environment, and 2. Methods of processing this information, i.e. producing new solutions and new behaviors based on this knowledge.

The first part (knowledge) is very large. It fills most of the memory and remains relatively constant (you remember your life, history and you can only fill it by what was in the past). The second part (methods for deciding, producing solutions based in your knowledge) is relatively small and constantly changing because of new information, facts and life experiences.

However, the most important part of a human soul can be written without any problem now. Industry is producing cheap micro-video recorders as small as a penny, microphones at grain size, and micro-sensors for vital signs (breathing, palpitation, blood pressure, skin resistance, perspiration, movement of body parts, etc.). These measurements allow for easy recording of not only the physical state, but of his moral state (joy, pleasure, grief, trouble, anxiety, nervousness, etc). For example, lie detectors are able to define not only the state of a man, but also the truth of his words. Now we can measure and record brain commands and we can produce small cards with four gigabytes of memory.

It would be easy to attach a video recorder and microphone to a man's forehead and then attach sensors to the body and record all that he sees, hears, speaks, his feelings, reactions, and activity. And then re-write this information into a personal hard drive (long-term memory of high capacity storage) at the end of each day. As a result, there is a record of the most important part our soul—history of life, feelings, environment, behaviors and actions. This would be more detailed than what is captured by the real man, because the humans forget many facts, feelings, emotions, and personal interactions. The electronic memory would not forget anything in the past. It would not forget any person or what they were doing.

But what about the second smaller part of the human soul—producing solutions based on personal knowledge—perhaps asks the meticulous reader.

This could be restored by using past information from the real man in similar situations. Moreover, an electronic man could analyze more factors and data in order to throw-out and exclude actions and emotions that happened under bad conditions. The electronic man (named E-being in my previous works) would have a gigantic knowledge base and could in a matter of second (write to his brain) produce the right answer, much faster than his biological prototype. That means he would not have the need for the second smaller part of memory.

Considering the environment and friends, the following is an important part of a man's soul: his relationship with parents, children, family, kin, friends, known people, partners and enemies. This part of his soul will be preserved more completely than even his prototype. Temporary factors will not influence his relationship with his enemy and friends as would happen with his former prototype.

There is one problem which may be troubling for some: if we were to record every part of a person's life, how do we keep intimate moments a secret? There are (will be) ways to protect private information which could be adapted from current usage, for example, the use of a password (known only by you). Also there may be some moments you choose not to record information or decide to delete the information from memory.

The offered system may become an excellent tool for defense again lies and false accusations. You may give the password in one given moment of your life, which proves your alibi or absence from the accusations.

Some people want to have better memory. Video takes 95% of storage capacity, sound takes 4% and the rest takes 1%. In usual situations, video can record only separate pictures, sound only when it appears. This type of recording practice decreases the necessary memory by tens of times. But every 1.5-2 years chip storage capacity doubles. There are systems which will compress the information and then may select to record the most important information (as is done in the human brain). During your life, the possibility to record all information will be available for all people. This type of recording apparatus will be widely available and inexpensive. It's possible now. The most advanced video recorder or DVD writes more information than a CD.

This solution (recording of human souls) is possible and must be solved quickly. By mass production (large productions) the apparatus will become inexpensive. The price will drop to about $300-1,000. If we work quickly we can begin recording and then more fully save our souls. The best solution is to begin recording in children when they become aware of “I”. But middle and older people should not delay. Unrecorded life periods may be restored by pictures, memories, notes, diaries and documents. Soul recovery will only be partial but it's better than nothing.

These records will also be useful in your daily life. You can restore recorded parts of your life, images of people, relatives, and then analyze and examine your actions for improvement.

### 6. Disadvantages of Biological Men and Biological Society

People understand Darwin's law, “survival of the fittest”. For a single person, this law is the struggle for his/her personal existence (life, well-being, satisfaction of requirements, pride, etc.). In a completely biological world built on Darwinian law the strongest survives and reaches his goal. Though they may be intelligent, humans are members of the animal world. They operate as any other animal in accordance with animal instincts of self-preservation. If one is poor, at first he struggles for food (currently half of world's population is starving), dwelling, and better living conditions. When one reaches material well-being, he may struggle for money, job promotion, reputation, renown, power, attractive women (men), and so on. Most people consider their activities (include official work) in only one way—what will I receive from it? Only a small number of people are concerned with the idea of sacrificing themselves to the well-being (seldom giving up their life) of society at large.

As a result, we see human history as a continuation of wars, dictatorships, and repression of people by power. Dictators kill all dissidents and opponents. Most people try to discriminate against opponents and play dirty against their enemy. There are murders, rapes, violence, robbery, underhand actions, fraud, and lying at all levels of society especially in lesser developed countries. Each person only cares for himself and his family and does not care how his actions effect other people or society.

Democratic countries try to cultivate a more civilized society. They create laws, courts, and have police. Dictator regimes, on the other hand, make only the law they want. I could give thousands of examples to verify this concept. But hundreds of millions of people are killed by war, aggressive campaigns, repressions, genocides, and thousands of criminals in the everyday world are a good illustration of this.

The human brain allows us to reach great success in science and technology. However, as a biological heritage, struggling for his INDIVIDUAL existence in a bloody, dangerous world, humans spend much of their resources on mutual extermination of intelligent beings. Moreover, humans have created ever powerful weapons (for example, nuclear and hydrogen bombs), which could wipe out humanity. In time, existence may depend on the volition of one man—perhaps the dictator of a nuclear state.

The second significant drawback to the biological body—is that it spends 99.99% of its effort and resources simply to support existence. Such as food, lodgings, clothing, sex, entertainment, relaxation, environment, ecological compatibility. Only a very small part is uses for scientific development and new ideas and technology. The reader may see something wrong here.

States use a parentage of their revenue for research into science and technology. This percent is used NOT for NEW ideas, but is used to commercialize modern processes All research is included in the state budget under the name, “Science and New Technology”. But much of this research has little relation to real new scientific progress. Even it the US, states spend only a small part of the assigned money on new science because state officers do not understand the research. People, organizations, and companies fight for a piece of the pie. Geniuses are rare and usually don't have the capacity to move forward because they must promote and pay for new ideas from their own empty packets.

Yet, science and technology has seen success. Most advancement (90%) was made recently in the 20th century, when governments started to finance a few scientific projects (compared with the millions of years of human existence). However, our current knowledge and new technologies are far from what we will eventually have. The first government of an industrialized country to understand and realize the leading role of new science and innovation will become powerful.

### 7. Electronic Society

The electronic society will be a society of clever electronic beings (or E-being, as they named in my articles). Most of the reasons and stimulus which incite men to crime, will be absent in E-beings. E-beings will not need food, shelter, sex, money, or ecology, which are the main factors in crime. E-people will not have intense infatuations or be distracted by behaviors, because they will have vast knowledge about the open electronic society. Their main work will be in science, innovations, and technologies. They will save their mental capacity for the production of chips and bodies, scientific devices, experimental equipments, space ships and space station, etc. They will need a number of robots, which do not need a big brain. It is likely they will award these robots better minds and memory. It is also likely that E-man will unite in a common distributed hyper-brain, which will become a sovereign of the Universe (God).

Nature is infinite and the development of a Super Brain (God) will not be limited. On the other hand, biological people will have limited mental capabilities. It will be difficult for them to image and predict the development and activity of Super beings, which we will generate.

Many, especially religious people, object because they say electronic beings will not have human senses such as love, sympathy, kindness, humanism, altruism, and the capacity to make mistakes, etc. E-beings are not people. Look back at human history. Human history shows that kindness played a very small role in human life. All human history is the history of human vices and human blood: struggle for power, authority, impact, money, riches, territory, and states. All human history is filled with fraud, underhanded actions, and trickery. Ordinary people were only playthings, flock of sheep for the tyrants and dictators.

Some people object that with an electronic face humans will loss the joy of sex, alcohol, narcotics, appreciation of art, beauty, nature, etc. My answer to this question is in my article “Science, Soul, Heaven, and Supreme Mind” (http://Bolonkin.narod.ru). The brief answer is that electronic humans will enjoy all this in a virtual world or virtual paradise. Time will run millions of times faster in the virtual World. E-man will spend a few seconds of real time and live millions of years in the paradise. He will enjoy any delight imaginable, include sex with any beautiful women (or handsome men), feel the emotions of any commander, leader, criminal, or even a dog.

### 8. Lot (Fortune) of Humanity

Biological humanity will be gradually transformed to electronic beings. Old people, when their biological bodies can not support their brains, will continue their existing in electronic bodies after death. They will become young, handsome, robust, and. Fertility in biological men will decrease. Birth-rates are less than death-rates in many civilized countries now (for example in France). Population growth is mainly supported by emigration from lesser developed countries. When education levels increase, birth-rates will fall.

For a time, biological and electronic people will exist together. However the distance between their capabilities will increase very quickly. Electronic people will reproduce (multiple) by coping, learn instantly, and will not need food or dwellings. They will work full days in any condition such as in space or on the ocean floor. They will gain new knowledge in a short time. They will pass this knowledge on to others who do not have enough time. The distance between biological and artificial intellects will reach a wide margin so that biological people will not understand anything about new science as monkeys do not understand multiplication now even after much explanation.

It is obvious, clever people will see that there will be a huge difference between the mental abilities of biological and electronic entities. They will try to transfer into electronic form and the ratio between biological and electronic entity will quickly change in electronic favor. A small number of outliers will continue to live in their biological body in special enclaves. They will not have industrial power or higher education and will begin to degrade.

Naysayers may promote laws against transferring into an electronic man (as cloning is forbidden now in some states). However, who would renounce immortality for themselves, especially while they are young and healthy? One may denounce immorality as blasphemy, but when your (parents, wife, husband, children) die, especially if you are near death yourself, one comes to understand that life is extremely important. The possibility to live forever, to gain knowledge that improves life, will also allow one to become a sovereign force in the Universe.

We summarize all said in the following steps and devices:

### Method

1. A method of writing and saving of Human Soul for man immortality comprising of steps:
   (a) positioning a micro video camera at a man head;
   (b) positioning a portable recorder at a man body;
   (c) positioning a portable memory at said man body;
   (d) positioning a microphone at said man body;
   (e) positioning a portable computer at said man body;
   (f) positioning a portable power source at said man body;
   (g) connecting said micro video camera, said microphone to said micro computer, said portable recorder, said power source and said portable memory;
   (h) programming said computer for writing needed data from said micro video camera, microphone whereby said portable recorder to said portable memory;
   (i) writing of views which said man see in during his life whereby said micro video camera, said portable computer, said portable recorder to said portable memory;
   (j) writing of sounds which person speaks and hears in during his life whereby said microphone, said portable computer, said portable recorder to said portable memory;
   (k) writing of time and dates whereby said portable computer, said portable recorder to said portable memory;
   (l) connecting said portable memory to a stationary long term high capability memory;
   (m)periodical rewriting an information from said portable memory to said stationary long term high capability personal memory;
   (n) read-protecting said information.
      Note: The simplest method can contain the parts of these steps, for example, writing only video and sound.
2. The Method of writing of Human Soul for man immortality as recited above comprising at least one of the following additional steps:
   (a) writing of brain impulses, oscillation, and a state of a man health and person physical conditions whereby micro sensors, said computer, said recorder to said portable memory;
   (b) writing of a person body position whereby micro sensors, said computer, said recorder to said portable memory;
   (c) writing of outer, environmental conditions around said man whereby micro sensors, said computer, said recorder to said portable memory;
   (d) writing person position at Earth surface whereby portable navigation system, said computer, said recorder to said portable memory;
   (e) permanently writing said conditions and information to said portable memory;
   (f) permanently writing said condition and information to said portable memory except slipping time;
   (g) periodically writing said condition and information to said portable memory;
   (h) writing said condition and information only in selected time to said portable memory;
   (i) data processing before writing to said portable memory;
   (j) data compressing before writing to said portable memory;
   (k) data coding of said information;
   (l) transferring of said information to stationary receiver.
3. The Method of writing of Human Soul for man immortality as recited above comprising at least one of the following additional steps:
   (a) writing of said man physical conditions includes at least one of the following: opening of man eyes, body temperature, blood press, heart palpitation, skin resistance, perspiration, skin temperature, respiration activity, mouth water, chill, rigor-like tremor, catarrh, muscle stress, blood mixture, pain;
   (b) writing of said outer conditions includes at least one of the following: air temperature, pressure, humidity, gas composition, smells, wind, illumination, radiation;
   (c) computer analyzing of said man physical and outer conditions;
   (d) informing about emergency situation said man and an outer authority;
   (e) long time saving whereby said high capability long-term memory.

### Installation (System)

4. An Installation utilized the Method of writing and saving of Human Soul for human immortality comprising devices:
   (a) micro video camera located at man head for writing of views which said man see in during his life;
   (b) microphone located at said man body for writing all sounds which person speaks and hears in during his life;
   (c) portable computer for data processing located at said man body and connected to said micro video camera and said microphone;
   (d) portable clock for writing of time located at man body and connected to said computer;
   (e) portable recorder located at man body and connected to said computer, said micro video camera and said microphone;
   (f) portable memory located at an said man body and connected to said recorder;
   (g) portable source located at said man body and connected to said computer, said micro video camera, said microphone and said memory;
   (h) stationary high capability long-term memory for rewriting of information from said portable memory;
   (i) communications located at said man body and connected said micro video camera, said microphone, said computer, said recorder, said memory, said power source in a common net.
      Note: The simplest instillations can contain the parts of these devices, for example, devices for writing video and sound.
5. The Installation recited above comprising at least one of the following devices:
   (a) micro sensors for writing of brain impulses, oscillation, fluctuation and a state of a man health and man physical conditions located at said person body and connected to said computer and to said portable recorder;
   (b) micro sensors for writing of man physical conditions located at said person body and connected to said computer and to said portable recorder;
   (c) micro sensors for writing of a state of a man health and person body positions located at said person body and connected to said computer and to said portable recorder;
   (d) devices for measure of outer environment conditions located at man body and connected to said computer;
   (e) devices of a portable navigation system for measure of a person position at Earth surface located at said man body and connected to said computer and said recorder;
   (f) devices for emergency communications located at man body and connected to said computer;
   (g) portable power source located at said person body and connected to said micro sensors, said devices, said computer, and said portable memory.
6. The Installation recited above comprising at least one of the following devices and features:
   (a) said micro video camera is located in man spectacles or on head decoration;
   (b) said computer is programmed chip for writing of said data in selected time, data processing and re-writing them to said high capability long-term data storage;
   (c) memory card used as said portable memory;
   (d) said physical condition sensors are at list one of the following: blood press, heart palpitation, skin resistance, perspiration, skin temperature, respiration activity, mouth water, chill, rigor-like tremor, catarrh, muscle stress, blood mixture, open eyes, pain;
   (e) said body position sensors are motion, acceleration, distance, angle, mutual position;
   (f) said devices for measuring of the outer environmental conditions includes at least one of the following values: air temperature, pressure, humidity, gas composition, smells, wind, illumination, radiation;
   (g) said power source is at least one of the following: chemical, solar, nuclear batteries;
   (h) said high capability long-term date storage is recorder used CD, DVD, type devices, memory carts.

I claim:

1. A method of writing and saving of Human Soul for man immortality comprising of steps:
(a) positioning a micro video camera at a man head;
(b) positioning a portable recorder at a man body;
(c) positioning a portable memory at said man body;
(d) positioning a microphone at said man body;
(e) positioning a portable computer at said man body;
(f) positioning a portable power source at said man body;
(g) connecting said micro video camera, said microphone to said micro computer, said portable recorder, said power source and said portable memory;
(h) programming said computer for writing needed data from said micro video camera, microphone whereby said portable recorder to said portable memory;
(i) writing of views which said man see in during his life whereby said micro video camera, said portable computer, said portable recorder to said portable memory;
(j) writing of sounds which person speaks and hears in during his life whereby said microphone, said portable computer, said portable recorder to said portable memory;
(k) writing of time and dates whereby said portable computer, said portable recorder to said portable memory;
(l) connecting said portable memory to a stationary long term high capability memory;
(m)periodical rewriting an information from said portable memory to said stationary long term high capability personal memory;
(n) read-protecting said information.

2. The Method of writing of Human Soul for man immortality as recited in claim 1 comprising at least one of the following additional steps:
(a) writing of brain impulses, oscillation, and a state of a man health and person physical conditions whereby micro sensors, said computer, said recorder to said portable memory;
(b) writing of a person body position whereby micro sensors, said computer, said recorder to said portable memory;
(c) writing of outer, environmental conditions around said man whereby micro sensors, said computer, said recorder to said portable memory;
(d) writing person position at Earth surface whereby portable navigation system, said computer, said recorder to said portable memory;
(e) permanently writing said conditions and information to said portable memory;
(f) permanently writing said condition and information to said portable memory except slipping time;
(g) periodically writing said condition and information to said portable memory;
(h) writing said condition and information only in selected time to said portable memory;
(m) data processing before writing to said portable memory;
(n) data compressing before writing to said portable memory;
(o) data coding of said information;
(p) transferring of said information to stationary receiver.

3. The Method of writing of Human Soul for man immortality as recited in claim 2 comprising at least one of the following additional steps:
(a) writing of said man physical conditions includes at least one of the following: opening of man eyes, body temperature, blood press, heart palpitation, skin resistance, perspiration, skin temperature, respiration activity, mouth water, chill, rigor-like tremor, catarrh, muscle stress, blood mixture, pain;
(b) writing of said outer conditions includes at least one of the following: air temperature, pressure, humidity, gas composition, smells, wind, illumination, radiation;
(c) computer analyzing of said man physical and outer conditions;
(d) informing about emergency situation said man and an outer authority;
(e) long time saving whereby said high capability long-term memory.

4. An Installation utilized the Method of writing and saving of Human Soul for human immortality comprising devices:
(a) micro video camera located at man head for writing of views which said man see in during his life;
(b) microphone located at said man body for writing all sounds which person speaks and hears in during his life;
(c) portable computer for data processing located at said man body and connected to said micro video camera and said microphone;
(d) portable clock for writing of time located at man body and connected to said computer;
(e) portable recorder located at man body and connected to said computer, said micro video camera and said microphone;
(f) portable memory located at an said man body and connected to said recorder;
(g) portable power source located at said man body and connected to said computer, said micro video camera, said microphone and said memory;
(h) stationary high capability long-term memory for rewriting of information from said portable memory;
(i) communications located at said man body and connected said micro video camera, said microphone, said computer, said recorder, said memory, said power source in a common net.

5. The Installation recited in claim 4 comprising at least one of the following devices:
(a) micro sensors for writing of brain impulses, oscillation, fluctuation and a state of a man health and man physical conditions located at said person body and connected to said computer and to said portable recorder;
(b) micro sensors for writing of person body positions located at said person body and connected to said computer and to said portable recorder;
(c) devices for measure of outer environment conditions located at man body and connected to said computer;
(d) devices of a portable navigation system for measure of a person position at Earth surface located at said man body and connected to said computer and said recorder;
(e) devices for emergency communications located at man body and connected to said computer;
(f) portable power source located at said person body and connected to said micro sensors, said devices, said computer, and said portable memory.

6. The Installation recited in claim 5 comprising at least one of the following devices and features:
(a) said micro video camera is located in man spectacles or on head decoration;
(b) said computer is programmed chip for writing of said data in selected time, data processing and re-writing them to said high capability long-term data storage;
(c) memory card used as said portable memory;
(d) said physical condition sensors are at list one of the following: blood press, heart palpitation, skin resistance, perspiration, skin temperature, respiration activity, mouth water, chill, rigor-like tremor, catarrh, muscle stress, blood mixture, open eyes, pain;
(e) said body position sensors are motion, acceleration, distance, angle, mutual position;
(f) said devices for measuring of the outer environmental conditions includes at least one of the following values: air temperature, pressure, humidity, gas composition, smells, wind, illumination, radiation;
(g) said power source is at least one of the following: chemical, solar, nuclear batteries;
(h) said high capability long-term date storage is recorder used CD, DVD, type devices, memory carts.
